(19) United States
(12) Patent Application Publication
(10) Pub. No.: US 2025/0372265 A1
(43) Pub. Date: Dec. 4, 2025

(54) METHOD OF ASSESSING RISK OF SUBJECT DEVELOPING BREAST CANCER

(71) Applicant: National Tsing Hua University, Hsinchu City (TW)

(72) Inventors: Li-An CHU, Hsinchu City (TW); Chao-Ying HUANG, Hsinchu City (TW); Jia-Rong LIN, Hsinchu City (TW)

(73) Assignee: National Tsing Hua University, Hsinchu City (TW)

(21) Appl. No.: 18/965,098

(22) Filed: Dec. 2, 2024

(30) Foreign Application Priority Data
May 30, 2024 (TW) 113120067

(51) Int. Cl. G16H 50/30; G06T 7/00; G16B 15/00; G16H 10/60; G16H 30/40

(52) U.S. Cl. CPC G16H 50/30; G06T 7/0012; G16B 15/00; G16H 10/60; G16H 30/40; G06T 2207/30024; G06T 2207/30068

(57) ABSTRACT

A method includes steps of: obtaining an original section image that is related to a subject and that includes cell-image portions; for each of the cell-image portions, determining a number of specific protein signals as a specific protein number, determining a number of specific chromosome signals as a specific chromosome number, and calculating a ratio of the specific protein number to the specific chromosome number as an individual protein-to-chromosome ratio; selecting N number of critical cell-image portions from among the cell-image portions according to the individual protein-to-chromosome ratios; and determining a risk of the subject developing breast cancer based on the specific protein number and the specific chromosome number determined for each of the N number of critical cell-image portions.

### CROSS-REFERENCE TO RELATED APPLICATION

This application claims priority to Taiwanese Invention patent application Ser. No. 11/312,0067, filed on May 30, 2024, and incorporated by reference herein in its entirety.

### FIELD

The disclosure relates to a method of assessing a risk of a subject developing breast cancer, and more particularly to a method of assessing a risk of a subject developing breast cancer based on a number of specific protein
signals and a number of specific chromosome signals shown in a section image related to the subject.

### BACKGROUND

Breast cancer diagnosis often involves mammography and/or pathological examination. In the conventional pathological examination, a medical professional visually inspects an immunostained tissue section of a patient. In particular, determination as to whether or not the patient has breast cancer is made based on a number of the human epidermal growth factor receptor 2 (HER2) and a number of the human chromosome 17 (Chr17) in cell(s) selected by the medical professional in the immunostained tissue section. However, there are a great number of cells in the immunostained tissue section, so it is not easy for the medical professional to select appropriate cell(s) from among the cells in the immunostained tissue section for diagnosis. At the same time, it takes a great amount of time to select cell(s) and make the aforesaid determination. That is to say, breast cancer diagnosis implemented by the conventional pathological examination may be inaccurate and inefficient.

### SUMMARY

Therefore, an object of the disclosure is to provide a method of assessing a risk of a subject developing breast cancer that can alleviate at least one of the drawbacks of the prior art.

According to the disclosure, the method includes steps of:
obtaining an original section image that is related to a tissue section of the subject, the original section image including a plurality of cell-image portions that correspond respectively to a plurality of cells of the tissue section;
for each of the cell-image portions,
   determining a number of specific protein signals shown in the cell-image portion as a specific protein number, the specific protein signals all indicating a specific protein,
   determining a number of specific chromosome signals shown in the cell-image portion as a specific chromosome number, the specific chromosome signals all indicating a specific chromosome, and
   calculating a ratio of the specific protein number to the specific chromosome number as an individual protein-to-chromosome ratio;
selecting N number of critical cell-image portions from among the cell-image portions, the N number of critical cell-image portions respectively corresponding to greatest N ones of the individual protein-to-chromosome ratios that are calculated respectively for the cell-image portions; and
determining the risk of the subject developing breast cancer based on the specific protein number and the specific chromosome number determined for each of the N number of critical cell-image portions.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is flow chart illustrating a method of assessing a risk of a subject developing breast cancer according to an embodiment of the disclosure.

FIG. 2 is a flow chart illustrating steps for determining a number of specific protein signals, a number of specific chromosome signals and an individual protein-to-chromosome ratio in the method according to an embodiment of the disclosure.

### DETAILED DESCRIPTION

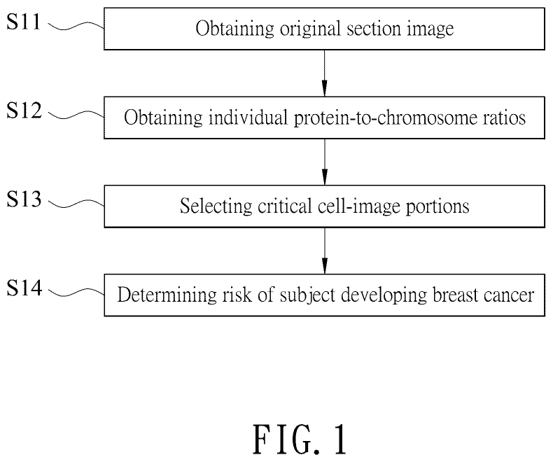

Referring to FIG. 1, a method of assessing a risk of a subject developing breast cancer according to an embodiment of the disclosure is illustrated. The method is to be implemented by a computing device. The computing device may be implemented to be a personal computer (e.g., a desktop computer, a laptop computer, a notebook computer or a tablet computer), a cloud server, a computing server, or a data server, but implementation thereof is not limited to what are disclosed herein and may vary in other embodiments. In this embodiment, the computing device includes an 11th Gen Intel® Core™ i7-1165G7 processor, 16GB RAM, and an NVIDIA® GeForce® MX350 GPU.

The method includes steps S11 to S14 as delineated below.

In step S11, the computing device obtains an original section image that is related to a tissue section of the subject. The original section image includes a plurality of cell-image portions that correspond respectively to a plurality of cells of the tissue section.

In step S12, for each of the cell-image portions, the computing device determines a number of specific protein signals shown in the cell-image portion as a specific protein number, determines a number of specific chromosome signals shown in the cell-image portion as a specific chromosome number, and calculates a ratio of the specific protein number to the specific chromosome number as an individual protein-to-chromosome ratio. The specific protein signals all indicate a specific protein, and the specific chromosome signals all indicate a specific chromosome. In this embodiment, the specific protein is the human epidermal growth factor receptor 2 (HER2), and the specific chromosome is the human chromosome 17 (Chr17).

It is worth to note that at the beginning, the tissue section of the subject has been stained by using techniques of the dual-color in-situ hybridization (DISH), which enable observation of signals from both HER2 and chromosome 17 centromere (CEP17), i.e., the specific protein signals and the specific chromosome signals shown in the original section image. Under DISH, the specific protein signals are presented with black dots, and the specific chromosome signals are presented in red.

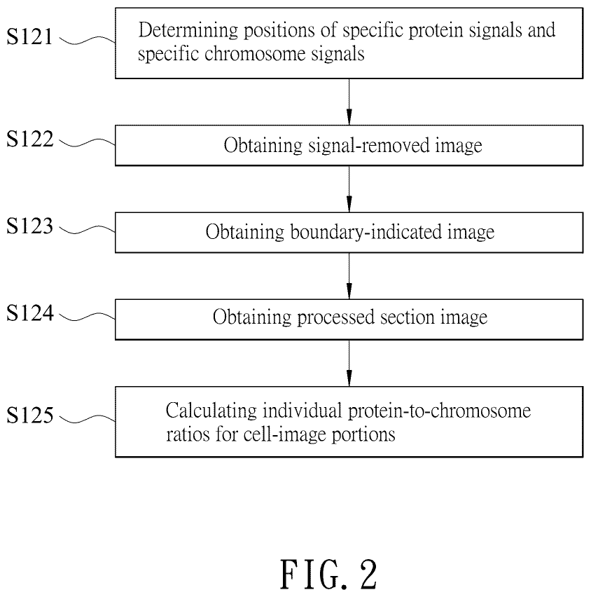

Specifically, step S12 includes sub-steps S121 to S125 illustrated in FIG. 2 and delineated below.

In sub-step S121, for each of the cell-image portions, the computing device identifies the specific protein signals and the specific chromosome signals, determines, for each of the specific protein signals, a position of the specific protein signal in the original section image, and determines, for each of the specific chromosome signals, a position of the specific chromosome signal in the original section image.

In this embodiment, identification of each of the specific protein signals and the specific chromosome signals is realized by using a technique of pixel classification, which is implemented by “scikit-learn”, a package or module of an programing language “Python”, as a random-forest-based pixel classification algorithm. More specifically, the “scikit-learn” is utilized to train a classifier for the specific protein signals and a classifier for the specific chromosome signals based on twenty different training section images. After that, the classifier for the specific protein signals is used to obtain an image in which only all of the specific protein signals are retained, and a position of each of the specific protein signals can be determined according to such image. Similarly, the classifier for the specific chromosome signals is used to obtain an image in which only all of the specific chromosome signals are retained, and a position of each of the specific chromosome signals can be determined according to such image.

In sub-step S122, the computing device removes all of the specific protein signals and the specific chromosome signals from the original section image to obtain a signal-removed image.

In sub-step S123, for each of the cell-image portions in the signal-removed image, the computing device detects a boundary of the cell-image portion to obtain a range of the cell-image portion. Then, for each of the cell-image portions in the signal-removed image, the computing device marks the boundary of the cell-image portion so as to obtain a boundary-indicated image.

In this embodiment, detection of the boundaries respectively of the cell-image portions is realized by utilizing a convolutional neural network (CNN) model with U-Net architecture, which is implemented by “StarDist”, a package or module of the programing language “Python”, as a pre-trained model “Versatile”. It is worth to note that when the pre-trained model “Versatile” is being utilized, downsampling is performed on the signal-removed image for an x-axis and a y-axis thereof at a reduced sampling rate that is 0.3 times an original sampling rate for the signal-removed image, so as to enhance precision of the detection of the boundaries. Moreover, a parameter related to a probability threshold for non-maximum suppression (NMS) post-processing provided by the package “StarDist” is set to be 0.375 for facilitating the detection of the boundaries. At the same time, a parameter related to an overlap threshold for NMS post-processing provided by the package “StarDist” is set to be 0 for preventing an adverse effect caused by overlapping cells on the detection of the boundaries (e.g., reduction in accuracy of detection).

It is worth to note that performing detection of the boundaries of the cell-image portions directly on the original section image may be interfered by the specific protein signals and the specific chromosome signals shown in the cell-image portions; for example, for a cell-image portion, the computing device may mistakenly identify a portion of one of the specific protein signals and the specific chromosome signals that is close to a boundary of the cell-image portion as the boundary of the cell-image portion; thus, removing all of the specific protein signals and the specific chromosome signals from the original section image prior to detecting the boundaries of the cell-image portions may improve accuracy of the detection of the boundaries, thereby improving accuracy of determining the specific protein numbers and the specific chromosome numbers for each of the cell-image portion.

In sub-step S124, the computing device adds all of the specific protein signals and the specific chromosome signals that were removed from the original section image into the boundary-indicated image so as to obtain a processed section image, wherein each of the specific protein signals and the specific chromosome signals is arranged at a position in the boundary-indicated image, and the position of each of the specific protein signals in the boundary-indicated image corresponds to the position of the specific protein signal in the original section image, and the position of each of the specific chromosome signals in the boundary-indicated image corresponds to the position of the specific chromosome signal in the original section image, i.e., each of the specific protein signals and the specific chromosome signals is arranged at an equivalent position (or called an identical position) of each of the boundary-indicated image and the original section image.

In sub-step S125, for each of the cell-image portions, the computing device counts a number of the specific protein signals, each of which has the position within the range of the cell-image portion before being removed, as the specific protein number, and counts a number of the specific chromosome signals, each of which has the position within the range of the cell-image portion before being removed, as the specific chromosome number. Thereafter, the computing device calculates the individual protein-to-chromosome ratios respectively for each of the cell-image portions.

In step S13, the computing device selects N number of critical cell-image portions from among the cell-image portions, where N is a positive integer. In this embodiment, N is 20, but is not limited thereto. The N number of critical cell-image portions respectively correspond to greatest N ones of the individual protein-to-chromosome ratios that are calculated respectively for the cell-image portions. In one embodiment, before selecting the N number of critical cell-image portions, the computing device sorts the cell-image portions in order of the individual protein-to-chromosome ratios from greatest to smallest.

In one embodiment, the computing device generates a critical-cell report. The critical-cell report contains, for each of the N number of critical cell-image portions, the specific protein number, the specific chromosome number and the individual protein-to-chromosome ratio. In addition, the computing device captures a part of the processed section image that includes only the N number of critical cell-image portions as at least one captured image. In one embodiment, said at least one captured image is a single image containing the N number of critical cell-image portions. In one embodiment, said at least one captured image includes N number of separate images respectively containing the N number of critical cell-image portions. In this way, a medical professional may perform further analysis on a result of assessment (i.e., assessing the risk of the subject developing breast cancer) based on the critical-cell report and said at least one captured image.

In step S14, the computing device determines the risk of the subject developing breast cancer based on the specific protein number and the specific chromosome number determined for each of the N number of critical cell-image portions.

Specifically, the computing device calculates a sum of the specific protein numbers determined for all of the N number of critical cell-image portions as a total protein number, calculates a sum of the specific chromosome numbers determined for all of the N number of critical cell-image portions as a total chromosome number, and calculates a ratio of the total protein number to the total chromosome number as a total protein-to-chromosome ratio. Additionally, the computing device calculates a quotient of the total protein number divided by N as an average protein number. Subsequently, the computing device determines the risk of the subject developing breast cancer based on the total protein-to-chromosome ratio and the average protein number.

Particularly, the computing device makes a first determination on whether the total protein-to-chromosome ratio is less than a first threshold (e.g., 2.0), makes a second determination on whether the average protein number is less than a second threshold (e.g., 4.0), makes a third determination on whether the average protein number is less than a third threshold (e.g., 6.0) that is greater than the second threshold, and determines the risk of the subject developing breast cancer based on a result of the first determination, a result of the second determination and a result of the third determination.

In response to determining that the total protein-to-chromosome ratio is not less than the first threshold and the average protein number is not less than the second threshold, the computing device determines that the risk of the subject developing breast cancer is relatively high.

In response to determining that the total protein-to-chromosome ratio is not less than the first threshold and the average protein number is less than the second threshold, the computing device determines that the risk of the subject developing breast cancer is relatively low.

In response to determining that the total protein-to-chromosome ratio is less than the first threshold and the average protein number is not less than the third threshold, the computing device determines that the risk of the subject developing breast cancer is relatively high.

In response to determining that the total protein-to-chromosome ratio is less than the first threshold and the average protein number is less than the third threshold but is not less than the second threshold, the computing device determines that the risk of the subject developing breast cancer is relatively low.

In response to determining that the total protein-to-chromosome ratio is less than the first threshold and the average protein number is less than the second threshold, the computing device determines that the risk of the subject developing breast cancer is relatively low.

Criteria used to determine the risk of the subject developing breast cancer are summarized in Table 1 below, where ΣHER2_signals represents the total protein number, ΣChr17_signals represents the total chromosome number,

$$\frac{\sum HER2\_signals}{\sum Chr 17 \_signals}$$

represents the total protein-to-chromosome ratio, and

$$\frac{\sum HER2\_signals}{N}$$

represents the average protein number.

TABLE 1

| | Result of |
|---|---|
| Criteria | assessment |
| $\frac{\sum HER2\_signals}{\sum Chr17\_signals} \geq 2. and \frac{\sum HER2\_signals}{N} \geq 4.0$ | High |
| $\frac{\sum HER2\_signals}{\sum Chr17\_signals} \geq 2. and \frac{\sum HER2\_signals}{N} < 4.$ | Low |
| $\frac{\sum HER2\_signals}{\sum Chr17\_signals} < 2. and \frac{\sum HER2\_signals}{N} \geq 6.$ | High |
| $\frac{\sum HER2\_signals}{\sum Chr17\_signals} < 2. and 6. > \frac{\sum HER2\_signals}{N} \geq 4.$ | Low |
| $\frac{\sum HER2\_signals}{\sum Chr17\_signals} < 2. and \frac{\sum HER2\_signals}{N} < 4.$ | Low |

It is worth to note that in one embodiment, after selecting N number of critical cell-image portions (hereinafter also referred to as the original N number of critical cell-image portions) from among the cell-image portions in step S13 and before determining the risk of the subject developing breast cancer in step S14, the computing device calculates the total protein-to-chromosome ratio for the original N number of critical cell-image portions, and determines whether the total protein-to-chromosome ratio is between 1.8 and 2.2. In response to determining that the total protein-to-chromosome ratio is between 1.8 and 2.2, the computing device selects additional N number of critical cell-image portions from among the cell-image portions, and combines the additional N number of critical cell-image portions and the original N number of critical cell-image portions selected in step S13 into 2N number of critical cell-image portions, where the additional N number of critical cell-image portions respectively correspond to greatest N ones of the individual protein-to-chromosome ratios; the greatest N ones of the individual protein-to-chromosome ratios, to which the additional N number of critical cell-image portions respectively correspond, are successive to the greatest N ones of the individual protein-to-chromosome ratios, to which the original N number of critical cell-image portions respectively correspond. The original N number of critical cell-image portions are those critical cell-image portions that correspond to the greatest to the Nth greatest individual protein-to-chromosome ratios, and the additional N number of critical cell-image portions are those critical cell-image portions that correspond to the (N+1) th greatest to the 2N^{th} greatest individual protein-to-chromosome ratios. In other words, there are 2N number of critical cell-image portions in total now. Then, a procedure flow of the method proceeds to step S14. Specifically, the computing device calculates a sum of the specific protein numbers determined for all of the 2N number of critical cell-image portions as the total protein number, calculates a sum of the specific chromosome numbers determined for all of the 2N number of critical cell-image portions as the total chromosome number, and calculates a ratio of the total protein number to the total chromosome number as the total protein-to-chromosome ratio. Additionally, the computing device calculates a quotient of the total protein number divided by 2N as the average protein number. Subsequently, the computing device determines the risk of the subject developing breast cancer based on the total protein-to-chromosome ratio and the average protein number.

Last but not least, the computing device outputs the critical-cell report, the captured image and a result of determination as to the risk of the subject developing breast cancer, so as to allow a medical professional to make a final determination about the risk of the subject developing breast cancer.

To sum up, the method of assessing a risk of a subject developing breast cancer according to the disclosure utilizes the computing device to automatically select critical cell-image portions from among cell-image portions in an original section image according to individual protein-to-chromosome ratios respectively of the cell-image portions, and to determine the risk of the subject developing breast cancer based on a specific protein number and a specific chromosome number determined for each of the critical cell-image portions. In this way, human labor may be saved, and efficiency of breast-cancer assessment may be improved under the power of multitasking of the computing device. It is worth to note that selection of the critical cell-image portions is implemented based on inspections of all cell-image portions in the original section image, and would not be influenced by the personal bias of a medical professional. Thus, breast-cancer assessment made by using the method according to the disclosure may be accurate and relatively objective.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method of assessing a risk of a subject developing breast cancer, the method comprising:
obtaining an original section image that is related to a tissue section of the subject, the original section image including a plurality of cell-image portions that correspond respectively to a plurality of cells of the tissue section;
for each of the cell-image portions,
determining a number of specific protein signals shown in the cell-image portion as a specific protein number, the specific protein signals all indicating a specific protein,
determining a number of specific chromosome signals shown in the cell-image portion as a specific chromosome number, the specific chromosome signals all indicating a specific chromosome, and
calculating a ratio of the specific protein number to the specific chromosome number as an individual protein-to-chromosome ratio;
selecting N number of critical cell-image portions from among the cell-image portions, the N number of critical cell-image portions respectively corresponding to greatest N ones of the individual protein-to-chromosome ratios that are calculated respectively for the cell-image portions; and
determining the risk of the subject developing breast cancer based on the specific protein number and the specific chromosome number determined for each of the N number of critical cell-image portions.

2. The method as claimed in claim 1, wherein determining the risk of the subject developing breast cancer includes:
calculating a sum of the specific protein numbers determined for all of the N number of critical cell-image portions as a total protein number;
calculating a sum of the specific chromosome numbers determined for all of the N number of critical cell-image portions as a total chromosome number;
calculating a ratio of the total protein number to the total chromosome number as a total protein-to-chromosome ratio;
calculating a quotient of the total protein number divided by N as an average protein number; and
determining the risk of the subject developing breast cancer based on the total protein-to-chromosome ratio and the average protein number.

3. The method as claimed in claim 2, wherein determining the risk of the subject developing breast cancer includes:
making a first determination on whether the total protein-to-chromosome ratio is less than a first threshold;
making a second determination on whether the average protein number is less than a second threshold; and
determining the risk of the subject developing breast cancer based on a result of the first determination and a result of the second determination.

4. The method as claimed in claim 3, wherein determining the risk of the subject developing breast cancer further includes:
making a third determination on whether the average protein number is less than a third threshold that is greater than the second threshold;
determining the risk of the subject developing breast cancer further based on a result of the third determination.

5. The method as claimed in claim 4, wherein determining the risk of the subject developing breast cancer further includes:
in response to determining that the total protein-to-chromosome ratio is not less than the first threshold and the average protein number is not less than the second threshold, determining that the risk of the subject developing breast cancer is relatively high;
in response to determining that the total protein-to-chromosome ratio is not less than the first threshold and the average protein number is less than the second threshold, determining that the risk of the subject developing breast cancer is relatively low;
in response to determining that the total protein-to-chromosome ratio is less than the first threshold and the average protein number is not less than the third threshold, determining that the risk of the subject developing breast cancer is relatively high;
in response to determining that the total protein-to-chromosome ratio is less than the first threshold and the average protein number is less than the third threshold but is not less than the second threshold, determining that the risk of the subject developing breast cancer is relatively low; and
in response to determining that the total protein-to-chromosome ratio is less than the first threshold and the average protein number is less than the second threshold, determining that the risk of the subject developing breast cancer is relatively low.

6. The method as claimed in claim 1, further comprising:
for each of the specific protein signals, determining a position of the specific protein signal in the original section image;
for each of the specific chromosome signals, determining a position of the specific chromosome signal in the original section image;
removing all of the specific protein signals and the specific chromosome signals from the original section image to obtain a signal-removed image; and
for each of the cell-image portions in the signal-removed image, detecting a boundary of the cell-image portion to obtain a range of the cell-image portion,
wherein determining the specific protein number for each of the cell-image portions is to count a number of the specific protein signals, each of which has the position within the range of the cell-image portion before being removed, as the specific protein number, and
wherein determining the specific chromosome number for each of the cell-image portions is to count a number of the specific chromosome signals, each of which has the position within the range of the cell-image portion before being removed, as the specific chromosome number.

7. The method as claimed in claim 6, further comprising:
for each of the cell-image portions in the signal-removed image, marking the boundary of the cell-image portion so as to obtain a boundary-indicated image.

8. The method as claimed in claim 7, further comprising, after detecting a boundary of the cell-image portion:
for each of the cell-image portions, adding all of the specific protein signals and the specific chromosome signals that were removed from the original section image into the boundary-indicated image so as to obtain a processed section image, wherein each of the specific protein signals and the specific chromosome signals is arranged at a position in the boundary-indicated image, the position of each of the specific protein signals in the boundary-indicated image corresponds to the position of the specific protein signal in the original section image, and the position of each of the specific chromosome signals in the boundary-indicated image corresponds to the position of the specific chromosome signal in the original section image.

9. The method as claimed in claim 8, further comprising:
generating a critical-cell report that contains, for each of the N number of critical cell-image portions, the specific protein number, the specific chromosome number and the individual protein-to-chromosome ratio.

10. The method as claimed in claim 9, further comprising:
capturing a part of the processed section image that includes only the N number of critical cell-image portions as at least one captured image.

11. The method as claimed in claim 10, further comprising:
outputting the critical-cell report, the at least one captured image and a result of determination as to the risk of the subject developing breast cancer.

12. The method as claimed in claim 8, further comprising:
capturing a part of the processed section image that includes only the N number of critical cell-image portions as at least one captured image.

13. The method as claimed in claim 1, further comprising:
generating a critical-cell report that contains, for each of the N number of critical cell-image portions, the specific protein number, the specific chromosome number and the individual protein-to-chromosome ratio.

14. The method as claimed in claim 1, further comprising, before selecting N number of critical cell-image portions, sorting the cell-image portions in order of the individual protein-to-chromosome ratios from greatest to smallest.

15. The method as claimed in claim 1, further comprising, subsequent to selecting N number of critical cell-image portions and prior to determining the risk of the subject developing breast cancer:
calculating a sum of the specific protein numbers determined for all of the N number of critical cell-image portions as a total protein number;
calculating a sum of the specific chromosome numbers determined for all of the N number of critical cell-image portions as a total chromosome number;
calculating a ratio of the total protein number to the total chromosome number as a total protein-to-chromosome ratio;
determining whether the total protein-to-chromosome ratio is between 1.8 and 2.2; and
in response to determining that the total protein-to-chromosome ratio is between 1.8 and 2.2,
selecting additional N number of critical cell-image portions from among the cell-image portions, and
combining the additional N number of critical cell-image portions and the N number of critical cell-image portions previously selected into 2N number of critical cell-image portions, where the additional N number of critical cell-image portions respectively correspond to greatest N ones of the individual protein-to-chromosome ratios successive to the greatest N ones of the individual protein-to-chromosome ratios, to which the N number of critical cell-image portions respectively correspond,
wherein determining the risk of the subject developing breast cancer includes
calculating a sum of the specific protein numbers determined for all of the 2N number of critical cell-image portions as another total protein number,
calculating a sum of the specific chromosome numbers determined for all of the 2N number of critical cell-image portions as another total chromosome number,
calculating a ratio of the another total protein number to the another total chromosome number as another total protein-to-chromosome ratio,
calculating a quotient of the another total protein number divided by 2N as an average protein number, and
determining the risk of the subject developing breast cancer based on the another total protein-to-chromosome ratio and the average protein number.
